(19) United States
(12) Patent Application Publication
(10) Pub. No.: US 2008/0262409 A1
(43) Pub. Date: Oct. 23, 2008

(54) HIGH FLOW RATE DISPOSABLE CASSETTE HEAT EXCHANGER

(76) Inventors: Joel Brian Derrico, Atlanta, GA (US); Steven Douglas Richeson, Decatur, GA (US)

Correspondence Address: KEVIN J. MACK, 242 CURTNER AVE SUITE N, PALO ALTO, CA, 94306, US

(21) Appl. No.: 11/947,193

(22) Filed: Nov. 29, 2007

Related U.S. Application Data
(60) Provisional application No. 60/913,528, filed on Apr. 23, 2007.

(51) Int. Cl. A61N 1/30; B21D 53/02; F25B 29/00

(52) U.S. Cl. 604/19; 165/61; 29/890.03

(57) ABSTRACT

A system and method for manufacturing a heat exchanger is disclosed. In some embodiments, the heat exchanger comprises a casing with a serpentine pathway, a membrane enclosed by the casing, an inlet tube or value for fluid to enter the heat exchanger, and an outlet tube or value for fluid to exit the heat exchanger. In other embodiments, the method comprises attaching a plurality of tubes or valves to a flexible container, creating an asymmetric passage in a rigid shell, enclosing the flexible container within the shell, and sealing the shell.

### CROSS REFERENCE TO RELATED APPLICATIONS

This application claims priority to U.S. Provisional Application Ser. No. 60/913,528 entitled “High Flow Rate Disposable Cassette Heat Exchanger,” filed Apr. 23, 2007, and incorporated herein by reference.

### FIELD OF THE INVENTION

The invention relates generally to systems and methods for injecting a fluid into a patient, and more particularly, to regulating the temperature of the fluid.

### BACKGROUND

Medical practitioners inject fluids into patients for a variety of reasons. For example, fluid is injected into a patient during an infusion or perfusion. Perfusion is the medical process of injecting fluid through a patient's organs or biological tissue. Generally, a medical practitioner performs a perfusion by inserting hollow flexible tubes, or catheters, into a patient and connecting the catheters to a pump. The pump regulates the flow of fluid through the catheters to a target region of the patient and a thermal device regulates the temperature of the fluid. Although typically performed with the target region open to the operating room environment, perfusion may also be performed with the target region enclosed with sutures.

Conventional devices for performing medical perfusions suffer from several shortcomings. First, commercial purpose-built devices are not readily available and ad-hoc solutions are not robust and tend to malfunction during extended perfusion sessions. For example, in continuous perfusion applications, such as Intraperitoneal Hyperthermic Chemotherapy (IPHC), fluid is cycled between the device and the patient for several hours. During such applications, the fluid flow rate may reach as high as 2000 milliliters per minute, which strains ad-hoc perfusion devices and renders them unreliable. Moreover, ad-hoc perfusion devices typically cannot reliably withstand the high internal pressure and thermal variation generated by momentary occlusions sometimes completely blocking the fluid circuit during extended perfusion sessions.

Second, conventional perfusion devices do not regulate fluid temperature with enough precision for many medical applications. For example, temperature regulation during IPHC will ideally be within +/−0.1 degrees Celsius, regardless of the fluid flow rate. Conventional perfusion devices do not generally deliver such precision, especially over extended perfusion sessions and over a variation in the flow rate through the perfusion circuit.

Finally, the temperature regulation of conventional perfusion devices typically deteriorates with variable fluid flow rates, causing fluid temperature fluctuations over time. These temperature fluctuations limit the usefulness of conventional perfusion devices in many medical applications, especially those such as IPHC that require a high level of temperate regulation throughout the entire perfusion session.

Thus, what is needed is a system and corresponding method for medical perfusion that alleviates some or all of the aforementioned shortcomings.

### BRIEF SUMMARY

A system and method for manufacturing a heat exchanger is disclosed. In some embodiments, the heat exchanger comprises a casing with a serpentine pathway, a membrane enclosed by the casing, an inlet value for fluid to enter the heat exchanger, and an outlet value for fluid to exit the heat exchanger. In other embodiments, the method comprises attaching a plurality of valves to a flexible container, creating an asymmetric passage in a rigid shell, enclosing the flexible container within the shell, and sealing the shell.

### BRIEF DESCRIPTION OF THE DRAWINGS

For a detailed description of exemplary embodiments of the invention, reference will now be made to the accompanying drawings in which:

FIG. 1 illustrates a heat exchanger constructed in accordance with embodiments of the invention;

FIG. 2 depicts an exemplary membrane used with the heat exchanger of FIG. 1 in accordance with embodiments of the invention;

FIG. 3 depicts the exterior casing of the heat exchanger of FIG. 1 in greater detail;

FIG. 4 illustrates the membrane of FIG. 2 enclosed by the exterior casing of FIG. 3 in accordance with embodiments of the invention;

FIG. 5 show the heat exchanger of FIG. 1 inserted into an infusion or perfusion device in accordance with embodiments of the invention;

FIG. 6 illustrates a cross sectional diagram before and while fluid is passed through the heat exchanger of FIG. 1 in accordance with embodiments of the invention; and

FIG. 7 illustrates an exemplary process for manufacturing and using the heat exchanger of FIG. 1 in accordance with embodiments of the invention.

### NOTATION AND NOMENCLATURE

In the following discussion and in the claims, the terms “including” and “comprising” are used in an open-ended fashion, and thus should be interpreted to mean “including, but not limited to”. Also, the term “couple, “couples,” or “coupled” is intended to mean either an indirect or direct electrical or communicative connection. Thus, if a first device couples to a second device, that connection may be through a direct connection, or through an indirect connection via other devices and connections. In addition, the term “data source” should be interpreted to mean any source of data. For example, a database storing information created by two or more entities represents a plurality of data sources.

### DETAILED DESCRIPTION

In this disclosure, numerous specific details are set forth to provide a sufficient understanding of the present invention. Those skilled in the art, however, will appreciate that the present invention may be practiced without such specific details. In other instances, well-known elements have been illustrated in schematic or block diagram form in order not to obscure the present invention in unnecessary detail. Additionally, some details have been omitted inasmuch as such details are not considered necessary to obtain a complete understanding of the present invention, and are considered to be within the understanding of persons of ordinary skill in the relevant art. It is further noted that all functions described herein may be performed in either hardware or software, or a combination thereof, unless indicated otherwise.

The following discussion is also directed to various embodiments of the invention. Although one or more of these embodiments may be preferred, the embodiments disclosed should not be interpreted, or otherwise used, as limiting the scope of the disclosure, including the claims, unless otherwise specified. In addition, one skilled in the art will understand that the following description has broad application, and the discussion of any embodiment is meant only to be illustrative of that embodiment, and not intended to suggest that the scope of the disclosure, including the claims, is limited to that embodiment.

FIG. 1 illustrates a heat exchanger 100 constructed in accordance with embodiments of the invention. As shown in FIG. 1, the heat exchanger 100 comprises an exterior casing 102 with a serpentine main passageway 104, an inlet tube 106, and an outlet tube 108. Fluid may enter the heat exchanger 100 through the inlet tube 106, flow through the main passageway 104, and exit through the outlet tube 108. The main passageway 104, inlet tube 106, and outlet tube 108 are preferably asymmetric to mechanically manipulate the amount of time the fluid remains within specific sections of the heat exchanger 100, thereby enabling the heat exchanger 100 to regulate the temperature of the fluid with a high degree of precision, specifically at high flow rates. The main passageway 104, from inlet tube 106 to outlet tube 108, preferably widens as the fluid progresses through the heat exchanger 100. This gradual increase in passage diameter allows the internal pressure of the heat exchanger 100 to be regulated by the backpressure at outlet tube 108 instead of the pressure at the inlet tube 104. This design also reduces the flow rate of the fluid as it progresses through the heat exchanger 100, thereby increasing the opportunity for the heat exchanger 100 to regulate fluid temperature.

As can be appreciated by one of skill in the art, the heat exchanger 100 may be used as part of a medical infusion or perfusion device, such as the device described in U.S. patent application Ser. No. 11/875,831 entitled “System and Method for Regulating the Temperature of a Fluid Injected into a Patient,” filed Oct. 20, 2007, and incorporated herein by reference. In addition, in at least some embodiments, the heat exchanger 100 is designed for single patient use and is removable and/or disposable. Thus, the heat exchanger 100 may be removed from the infusion or perfusion device after use and discarded. Although not explicitly shown in FIG. 1, the heat exchanger 100 may also comprise a membrane for carrying fluid through the heat exchanger 100. The exterior casing 102 encloses the membrane which resides between the fluid and the casing.

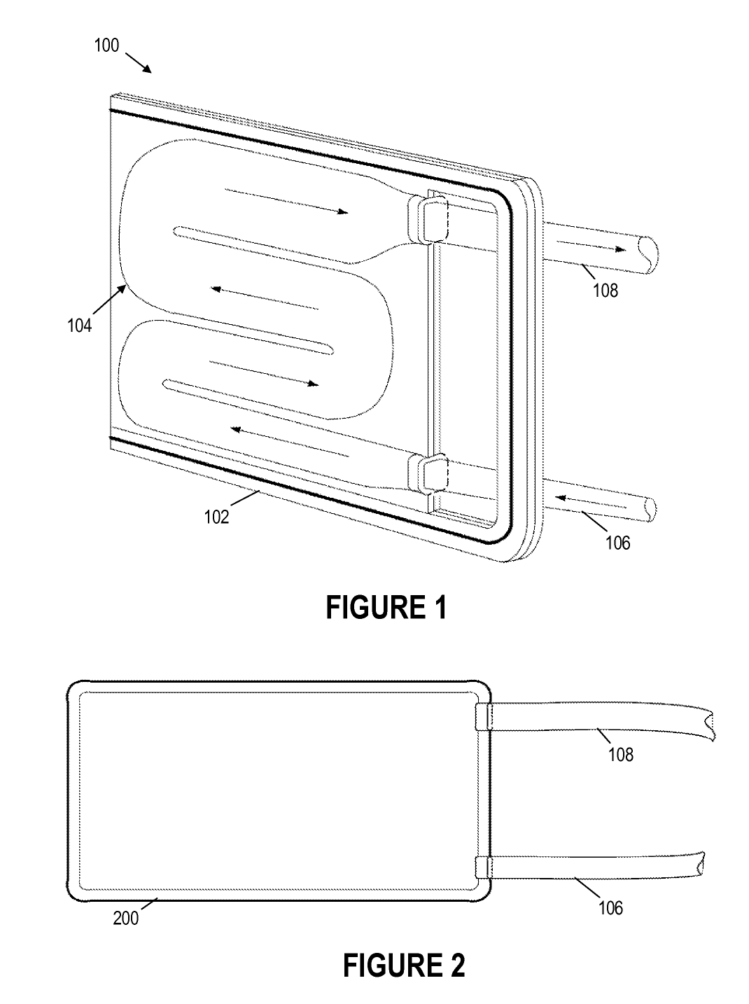

FIG. 2 depicts an exemplary membrane 200 used in conjunction with the heat exchanger of FIG. 1 in accordance with embodiments of the invention. During manufacture of the heat exchanger 100, the inlet tube 106 and outlet tube 108 are preferably attached to the edge of the membrane 200. Although the membrane 200 is preferably a thin-film plastic bag, rectangular in shape, any type of flexible container may also be used. The membrane 200 may be made of any type of plastic or polymer, such as polyvinylchloride (PVC). In addition, the membrane 200 preferably has a uniform thickness of between 4 and 5 mils and a known thermal conductivity so that the amount of thermal energy passed through the membrane to the fluid is known.

Ideally, the membrane 200 is capable of consistently transferring a determinable amount of energy to the fluid without material deformation or deterioration. In addition, although both the inlet tube 106 and the outlet tube 108 have an arbitrary length, the internal diameter of the outlet tube 108 is preferably greater than that of the inlet tube 106 to reduce backpressure within the heat exchanger 100. This backpressure could potentially rupture the thin-film plastic membrane if left unchecked. In addition, the wall thickness of the outlet tube 108 is preferably greater than the inlet tube 106 to provide increased insulation and reduced heat flux when the fluid exits the heat exchanger 110. Thus, both the internal and external diameter of the outlet tube may be greater than the inlet tube.

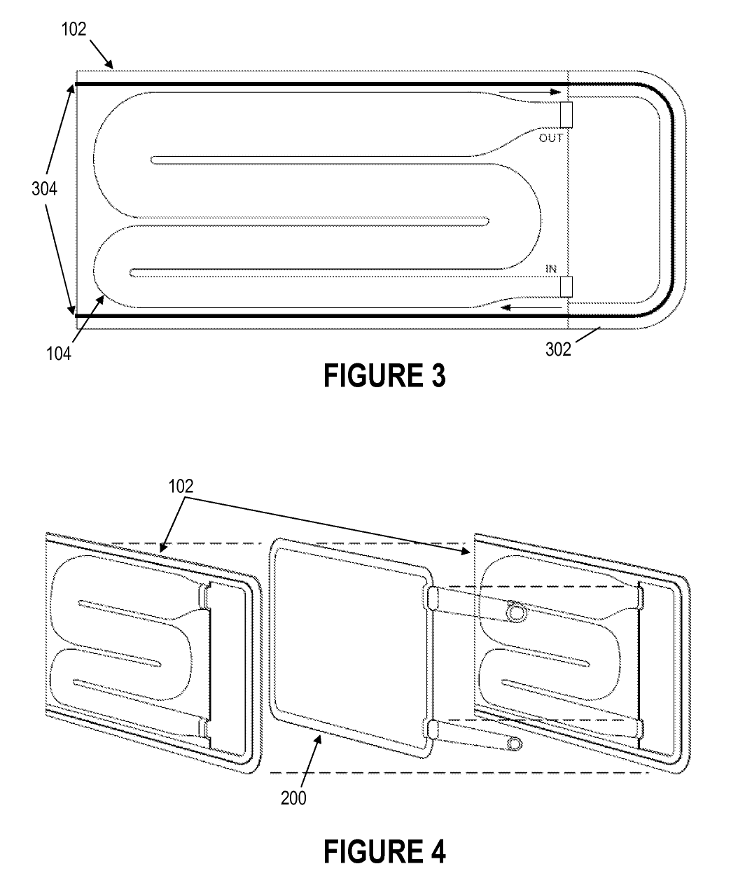

FIG. 3 depicts the exterior casing of the heat exchanger of FIG. 1 in greater detail. As shown in FIG. 3, the exterior casing 102 possesses the wide asymmetrical serpentine pathway 104. The exterior casing 102 preferably comprises a rigid, plastic frame. The frame consists of two halves, where the shape of the pathway 104 is mirrored on both halves of the frame so that when the halves are sandwiched together they form a channel. The frame also preferably incorporates a handle 302 to manipulate the insertion and extraction of the heat exchanger 100 from an infusion or perfusion device. In addition, the frame preferably has raised ribs 304 to strengthen the frame and act as insertion guide rails. The guide rails are preferably keyed so that the heat exchanger 100 may only be inserted in the correct orientation into the slotted opening of the infusion or perfusion device. The frame also preferably includes labels identifying the inlet or outlet tubes, as well as the correct direction of fluid flow.

FIG. 4 illustrates the membrane 200 enclosed by the exterior casing 102 in accordance with embodiments of the invention. As shown in FIG. 4, the heat exchanger 100 is preferably formed by positioning the membrane 200 between the halves of the exterior casing 102 with the inlet and outlet tubes facing the handle. The halves of the exterior casing 102 are then closed around the membrane 200 so that the two halves bond together, firmly encapsulating the membrane. The halves of the casing 102 may be thermal bonded, attached with a sealant, or coupled by any other means for securing the casing 102. Thus, the membrane resides between the surface layers of the exterior casing 102. The membrane 200 and/or the exterior casing 102 may be transparent to enable optical measurement of fluid temperature at multiple positions in the serpentine pathway.

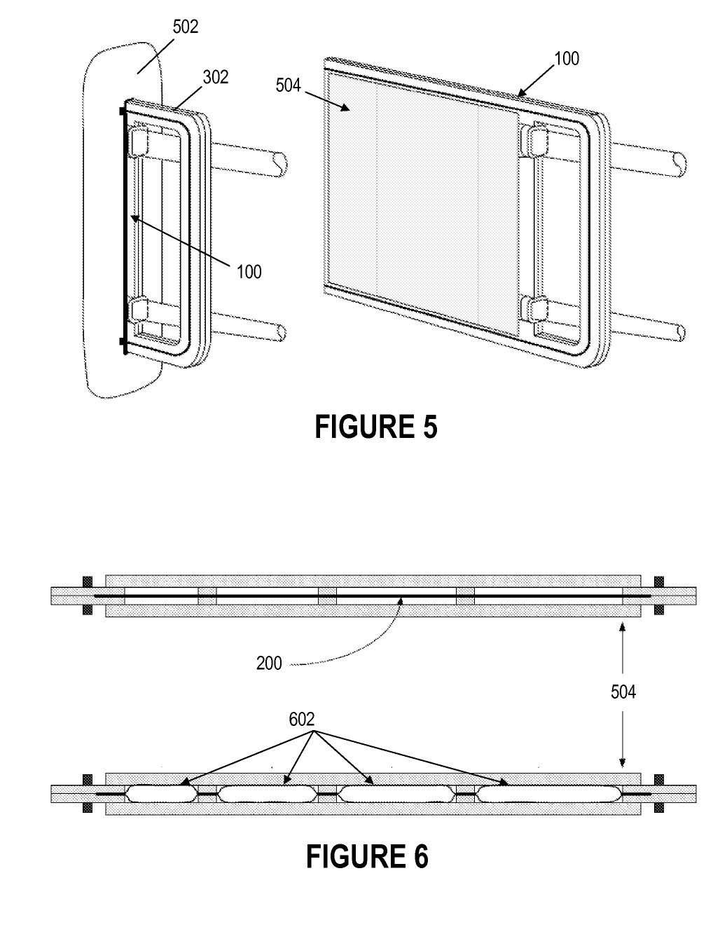

FIG. 5 show the heat exchanger of FIG. 1 inserted into an infusion or perfusion device in accordance with embodiments of the invention. As shown in FIG. 5, the heater exchanger 100 is inserted into an infusion or perfusion device 502 by way of the handle 302. After insertion, the heat exchanger 100 is firmly sandwiched between temperature control plates 504, which are located on both sides of the heat exchanger 100. The temperature control plates 504, which are integrated into the infusion or perfusion device 502, are designed to firmly hold the heat exchanger 100 during operation so that high internal fluid pressure will not separate the halves of the external casing 102.

FIG. 6 illustrates a cross sectional diagram before and while fluid is passed through the heat exchanger in accordance with embodiments of the invention. The top portion of FIG. 6 illustrates the heat exchanger before fluid is introduced, and the bottom portion of FIG. 6 show the heat exchanger while fluid if flowing through the unit. As previously discussed, the fluid is pumped into the heat exchanger through the inlet tube. The fluid pressure buildup causes the membrane 200 to distend in the shape of the asymmetric serpentine passageway, creating a continuous flat tube 602 that makes contact with the temperature control plates 504. The width of the flat tube 602 in the passageway is preferably much larger than the distended depth, thereby creating a large surface area to fluid volume ratio in the passageway. Heat may be transferred from the temperature control plates 504 through conduction, radiation, and/or convection between the temperature control plates 504 and the fluid within the flat tube 602. Upon completion of the infusion or perfusion procedure, the heat exchanger may be manually withdrawn from the slot using the attached handle and safely disposed.

As an added safety measure, the exterior casing of the heat exchanger is preferably notched to coincide with an optical presence detection circuit mounted within the infusion or perfusion device. If the optical circuit detects an improperly inserted heat exchanger, the device may become inoperable. The heat exchanger slot in the device is also preferably sealed to prevent liquid from entering the device in the event of a rupture in the membrane and includes a leak detection circuit with a liquid catch basin that automatically shuts down the device and the associated pump and sounds an alarm.

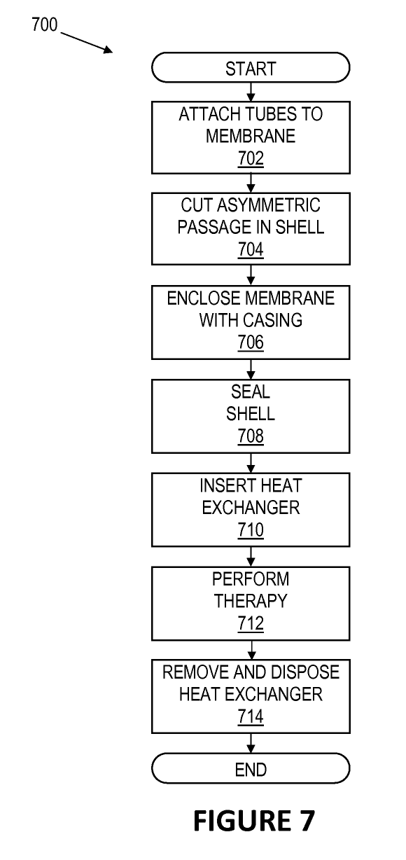

FIG. 7 illustrates an example process for manufacturing and using a heat exchanger in accordance with embodiments of the invention. The process 700 starts by attaching an inlet and outlet tube to a membrane, such as a thin-film, plastic membrane bag (702). An asymmetric passage may then be molded or cut from a shell (704). The passage may also gradually widen from beginning to end. The membrane may then be enclosed within the shell (706). The shell is preferably a rigid plastic casting capable of withstanding the thermal activity and pressure generated during infusion or perfusion therapy. The shell may then be sealed with the membrane inside to form a heat exchanger (708). The heat exchanger may then be inserted into an infusion or perfusion device (710) and the therapy may commence (712). During therapy, fluid is passed through the heat exchanger. After therapy ends, the heat exchange may be removed and disposed (714). A new heat exchanger may be inserted into the infusion or perfusion device and another therapy session may commence. Various steps of the process 700 may be added, removed, and reordered as desired. For example, before therapy commences, a sensor may determine if the heat exchanger is properly installed. If the heat exchanger is properly installed, the therapy may continue. If not, the infusion or perfusion device may become inoperable until the heat exchanger is properly installed.

The above discussion is meant to be illustrative of the principles and various embodiments of the present invention. Numerous variations and modifications will become apparent to those skilled in the art once the above disclosure is fully appreciated. For example, in addition to or in place of the handle and guide rails, the heat exchanger may comprise an electronic device the automatically feeds the heat exchanger into the infusion or perfusion device once placed in a slot designed for the heat exchanger. The electronic device may ensure that the heat exchanger is properly inserted to minimize human error. It is intended that the following claims be interpreted to embrace all such variations and modifications.

What is claimed is:

1. A heat exchanger, comprising:
a casing with a serpentine pathway;
a membrane enclosed by the casing;
an inlet value for fluid to enter the heat exchanger; and
an outlet value for fluid to exit the heat exchanger.

2. The heat exchanger of claim 1 wherein the serpentine pathway is asymmetric and gradually widens as fluid flows through the serpentine pathway.

3. The heat exchanger of claim 1 wherein pressure created by the flow of fluid through the pathway is directed away from a seam in the casing.

4. The heat exchanger of claim 1 wherein the outlet value has a larger internal diameter than the inlet value.

5. The heat exchanger of claim 1 wherein the outlet value has a larger external diameter than the inlet value.

6. The heat exchanger of claim 1 wherein the membrane distends to roughly the shape of the serpentine pathway when fluid flows through the heat exchanger.

7. The heat exchanger of claim 1 wherein the membrane is transparent and the heat exchanger further comprises a device to optically measure the temperature of the fluid.

8. The heat exchanger of claim 1 further comprising a handle that allows the heat exchanger to be inserted and removed from one of a medical perfusion device and a medical infusion device.

9. A method for manufacturing a heat exchanger, comprising:
attaching a plurality of valves to a flexible container;
creating an asymmetric passage in a rigid shell;
enclosing the flexible container within the shell; and
sealing the shell.

10. The method of claim 9 wherein attaching a plurality of valves comprises attaching an entry value for fluid to enter the heat exchanger and an exit value for fluid to exit the heat exchanger.

11. The method of claim 9 wherein creating an asymmetric passage comprises molding the passage in a plastic shell so that an internal dimension at an end of the passage is greater than an internal dimension at a beginning of the passage.

12. The method of claim 9 wherein sealing the shell comprises one of thermally bonding and chemically bonding the shell at a seam.

13. A heat exchanger, comprising:
an asymmetric means for controlling the flow fluid;
a flexible means for containing fluid;
a means for fluid to enter the heat exchanger; and
a means for fluid to exit the heat exchanger.

14. The heat exchanger of claim 1 wherein the asymmetric means for controlling the flow a fluid gradually widens as fluid flows through the asymmetric means.

15. The heat exchanger of claim 1 wherein pressure created by the flow of fluid through the asymmetric means for controlling the flow a fluid is directed away from a seam in the asymmetric means.

16. The heat exchanger of claim 1 wherein the means for fluid to exit the heat exchanger has a larger internal diameter than the means for fluid to enter the heat exchanger.

17. The heat exchanger of claim 1 wherein the means for fluid to exit the heat exchanger has a larger external diameter than the means for fluid to enter the heat exchanger.

18. The heat exchanger of claim 1 wherein the flexible means for containing fluid distends to roughly the shape of the asymmetric means for controlling the flow fluid when fluid flows through the heat exchanger.

19. The heat exchanger of claim 1 wherein the flexible means for containing fluid is transparent and the heat exchanger further comprises a means for optically measuring the temperature of fluid.

20. The heat exchanger of claim 1 further comprising a means for inserting and removing the heat exchanger from one of a means for performing a medical infusion and a means for performing a medical perfusion.
